(19) United States
(12) Patent Application Publication
(10) Pub. No.: US 2013/0103167 A1
(43) Pub. Date: Apr. 25, 2013

(54) KNEE JOINT

(71) Applicants: Chien-Wen Chen, New Taipei City (TW); Chien-Cheng Chen, New Taipei City (TW)

(72) Inventors: Chien-Wen Chen, New Taipei City (TW); Chien-Cheng Chen, New Taipei City (TW)

(21) Appl. No.: 13/658,906

(22) Filed: Oct. 24, 2012

(30) Foreign Application Priority Data
Oct. 25, 2011 (TW) 100220034

(51) Int. Cl. A61F 2/64

(52) U.S. Cl. 623/43

(57) ABSTRACT

A knee joint includes a knee member including a movable plate connected to and pressable toward a fixed seat, and an interspace formed between the fixed seat and the movable plate. An upper connecting unit disposed atop the knee member abuts against the movable plate. A pivot shaft is inserted threadedly into the knee member and is rotatable relative thereto. Pressing the upper connection unit against the movable plate constricts the interspace so that the pivot shaft is clamped tightly between the fixed seat and the movable plate, thereby restricting a relative rotation between the knee member and the pivot shaft. The movable plate is restored to its original position when unpressed.

### CROSS-REFERENCE TO RELATED APPLICATION

This application claims priority of Taiwanese Application No. 100220034, filed on Oct. 25, 2011, the disclosure of which is incorporated herein by reference.

### BACKGROUND OF THE INVENTION

1. Field of the Invention

The invention relates to a knee joint, more particularly to a prosthetic knee joint capable of providing effective resistance to excessive relative rotation between a knee member and a pivot shaft.

2. Description of the Related Art

For a physically disabled person wearing a prosthesis, the operation of a prosthetic knee joint that interconnects a prosthetic upper leg and a prosthetic lower leg is of paramount importance. For example, when a wearer of a prosthetic knee joint that does not have rotational resistance walks on uneven ground, the unevenness of the ground could cause the prosthetic knee joint to pivot excessively, thereby increasing the risk that the wearer could fall due to loss of balance when the prosthetic knee joint pivots.

Referring to FIG. 1, Taiwanese Utility Model No. M384644 discloses a prosthetic knee joint that includes a knee member 3, which has a fixture seat 31, a movable plate 32 integrally connected to the fixture seat 31, and a shaft-receiving hole 312 formed between the fixture seat 31 and the movable plate 32. A C-shaped sleeve 5 sheathes a pivot shaft 11, and the C-shaped sleeve 5 and the pivot shaft 11 are inserted together into the shaft-receiving hole 312. When the movable plate 32 abuts against the C-shaped sleeve 5 in the shaft-receiving hole 312, the rotation of the movable plate 32 relative to the pivot shaft 11 is restricted, thereby achieving a braking function. However, there is still a need to further improve the efficiency of the restriction of the relative movement between the movable plate 32 and the pivot shaft 11.

### SUMMARY OF THE INVENTION

Therefore, an object of the present invention is to provide a knee joint capable of providing effective resistance to excessive relative rotation between a knee member and a pivot shaft.

According to the present invention, a knee joint is adapted for connection to a prosthetic upper leg and a prosthetic lower leg. The knee joint includes a knee member, an upper connection unit, and a pivot shaft.

The knee member includes a fixed seat, a movable plate connected integrally to the fixed seat, a threaded hole formed between the fixed seat and the movable plate, and an interspace formed between the fixed seat and the movable plate that communicates with the threaded hole. The movable plate is pressable toward the fixed seat and can thereby reduce the size of the interspace.

The upper connecting unit is disposed on top of the knee member for connecting to the prosthetic upper leg and abuts against the movable plate.

The pivot shaft has a threaded portion inserted threadedly into the threaded hole such that the pivot shaft is rotatable relative to the knee member.

When the upper connection unit is pressed against the movable plate, the interspace is constricted so that the pivot shaft is clamped tightly between the fixed seat and the movable plate, thereby restricting a relative rotation between the knee member and the pivot shaft. The movable plate is restored to its original position when it is unpressed.

### BRIEF DESCRIPTION OF THE DRAWINGS

Other features and advantages of the present invention will become apparent in the following detailed description of the preferred embodiment with reference to the accompanying drawings, of which:

FIG. 1 is an exploded perspective view illustrating a conventional knee joint;

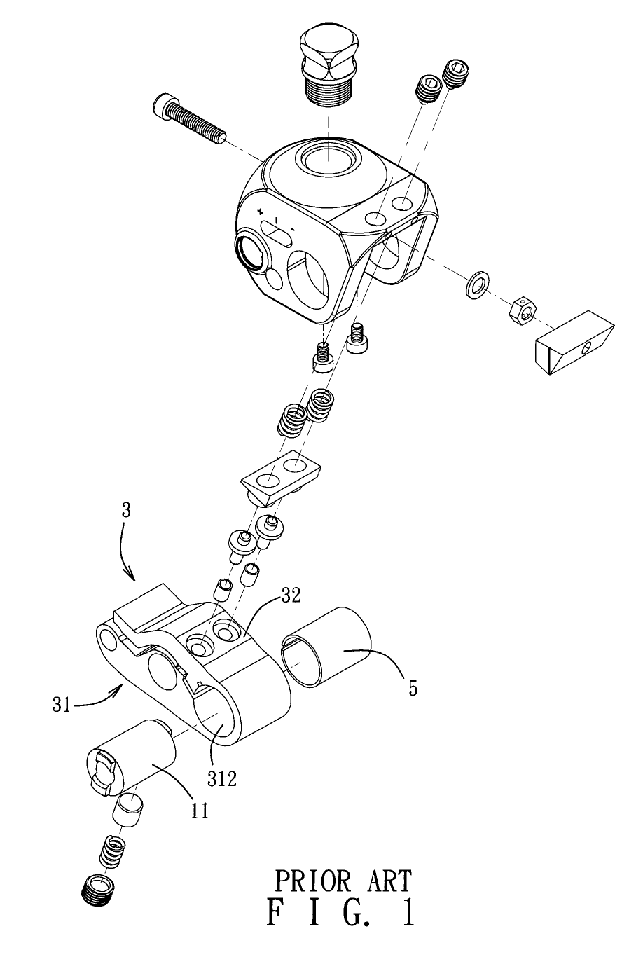

FIG. 2 is an exploded perspective view illustrating the preferred embodiment of a knee joint according to the present invention;

FIG. 3 is a side view of the preferred embodiment in an assembled state;

FIG. 4 is a sectional view of the preferred embodiment illustrating a brake block that is movable to adjust the size of an interspace between a fixed seat and a movable plate; and

FIG. 5 is an enlarged fragmentary sectional view illustrating the constriction of the interspace when the brake block biases the movable plate toward the fixed seat.

### DETAILED DESCRIPTION OF THE PREFERRED EMBODIMENT

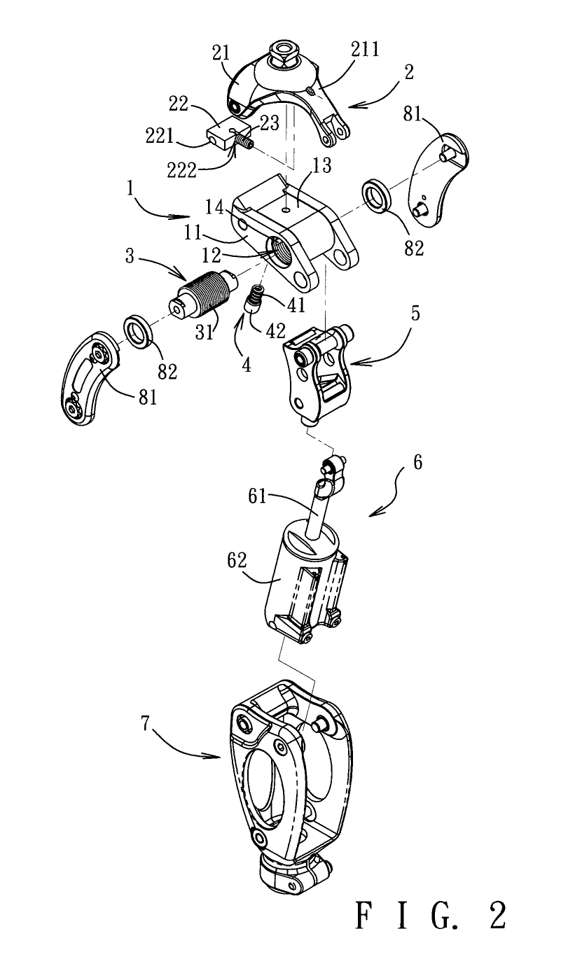

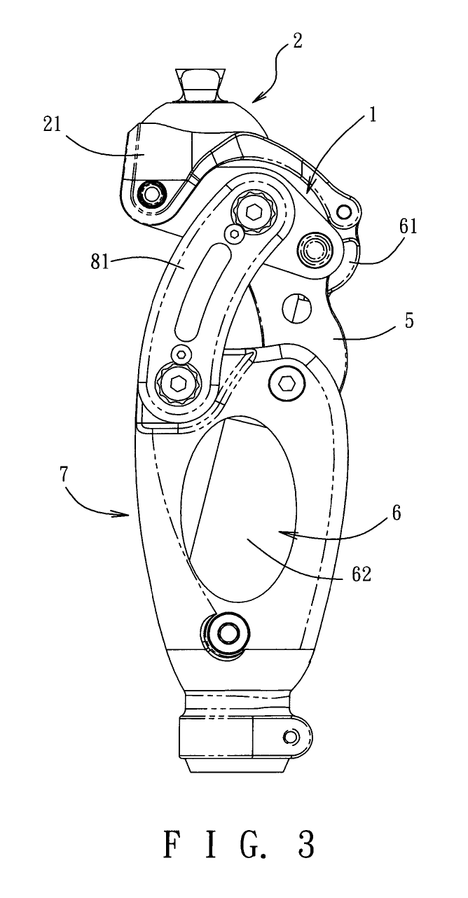

FIGS. 2 and 3 illustrate the preferred embodiment of a knee joint of the present invention adapted to connect to a prosthetic upper leg (not shown) and a prosthetic lower leg (not shown). The knee joint includes a knee member 1, an upper connecting unit 2 adapted to connect to the prosthetic upper leg, and a pivot shaft 3 adapted to connect to the prosthetic lower leg. Preferably, the knee joint further includes a restoring member 4, a damping device 6, a link member 5, two arcuate plates 81, and a connection seat 7.

The knee member 1 is disposed between the upper connecting unit 2 and the connection seat 7. The pivot shaft 3 is rotatably inserted into the knee member 1. The restoring member 4 is disposed within the knee member 1. The link member 5 is disposed between the knee member 1 and the connection seat 7, and has two opposite ends respectively pivotally connected to the knee member 1 and the connection seat 7. The arcuate plates 81 sandwich the link member 5 therebetween, and each arcuate plate 81 has two opposite end portions respectively pivotally connected to the knee member 1 and the connection seat 7. The end portion of the actuate plate 81 that is pivotally connected to the knee member 1 is fixed to and moves together with the pivot shaft 3. In this case, the arcuate plates 81 are symmetrically and respectively connected to the two sides of the knee member 1 and two sides of the connection seat 7. The damping device 6 is disposed within the connection seat 7, extends into the link member 5, and has two opposite ends respectively and pivotally connected to the upper connecting unit 2 and the connection seat 7.

Referring to FIGS. 2 to 5, the structure and the operation of the preferred embodiment of the knee joint according to the present invention are elaborated in the following detailed description.

The knee member 1 includes a fixed seat 11, a movable plate 13 connected integrally to the fixed seat 11, a threaded hole 12 formed between the fixed seat 11 and the movable plate 13, and an interspace 14 formed between the fixed seat 11 and the movable plate 13 that communicates with the threaded hole 12. The fixed seat 11 has a rear portion pivotally connected to the link member 5 and a front portion opposite to the rear portion that is connected to the upper connecting unit 2. The movable plate 13 is pressable toward the fixed seat 11, and when it is pressed, the size of the interspace 14 is reduced.

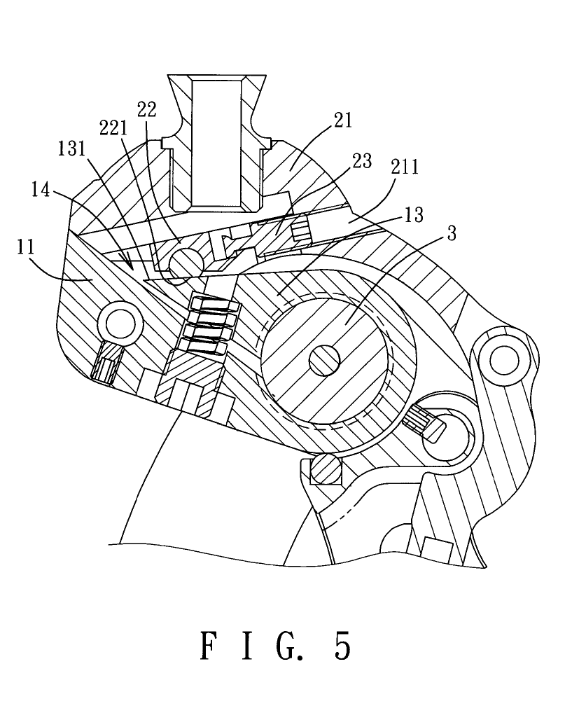

The upper connecting unit 2 abuts against the movable plate 13 and includes a connector 21 adapted for connecting to the prosthetic upper leg and a brake block 22 disposed between the connector 21 and the movable plate 13. In the preferred embodiment, a front portion of the connector 21 is connected to the front portion of the fixed seat 11 and a rear portion of the connector 21 is pivotally connected to the damping device 6. The brake block 22 has an abutment surface 221, and the movable plate 13 has a contact surface 131 in contact with the abutment surface 221. The size of the interspace 14 is adjusted by displacing the brake block 22 to move the movable plate 13 toward or away from the fixed seat 11. Preferably, the upper connecting unit 2 further includes a brake regulator 23 that extends through the connector 21, that connects to the brake block 22, and that can move the brake block 22 to adjust the size of the interspace 14. In the preferred embodiment, the brake regulator 23 is a regulating screw. The connector 21 has a threaded slot 211 for receiving the brake regulator 23. The brake block 22 further has a fixing hole 222 that securely receives one end of the brake regulator 23. In the preferred embodiment, the fixing hole 222 is circular. As shown in FIG. 5, when the brake regulator 23 is rotated, the brake regulator 23 moves backward and forward within the threaded slot 211, thereby shifting the brake block 22 in the same direction. Because the abutment surface 221 of the brake block 22 can be pressed to abut against the contact surface 131 of the movable plate 13, the wearer can adjust the size of the interspace 14 in accordance to the weight of the wearer by rotating the brake regulator 23.

The pivot shaft 3 has a threaded portion 31 inserted threadedly into the threaded hole 12 such that the pivot shaft 3 is rotatable relative to the knee member 1. When the upper connection unit 12 is pressed against the movable plate 13, the interspace 14 is constricted so that the pivot shaft 3 is clamped tightly between the fixed seat 11 and the movable plate 13, thereby restricting a relative rotation between the knee member 1 and the pivot shaft 3. Specifically, the moveable plate 13 is restored when it is unpressed. Compared to the prior art, the threaded relationship between the threaded portion 31 of the pivot shaft 3 and the threaded hole 12 of the knee member 1 in the present invention is able to increase friction therebetween, thereby effectively restricting the relative rotation between the knee member 1 and the pivot shaft 3.

The restoring member 4 includes a restoring spring 41 for restoring the movable plate 13 that is disposed in the fixed seat 11 and abuts against the movable plate 13. Preferably, the knee member 1 further includes a receiving aperture 15 to receive the restoring member 4. The restoring member 4 further includes a cap 42 that is secured to the receiving aperture 15. The restoring spring 41 has two ends that respectively abut against the cap 42 and the movable plate 13. Moreover, a spring force of the restoring spring 41 can restore the movable plate 13 to its original position.

The damping device 6 includes a piston rod 61 and a pneumatic cylinder 62. The piston rod 61 has two opposite ends, one of which is pivotally connected to the connector 21 of the upper connecting unit 2 and the other of which is movably inserted into the pneumatic cylinder 62. The pneumatic cylinder 62 is disposed within the connection seat 7 and has an end portion distal from the piston rod 61 that is pivotally connected to the connection seat 7. When the knee joint pivots, the damping device 6 primarily provides a cushioning effect. After the pressure is released, the damping device 6 restores the knee joint to the original position.

In this embodiment, the knee joint further has two journal bearings 82 that are respectively sleeved on two end portions of the pivot shaft 31 and that are respectively disposed between the threaded portion 31 of the pivot shaft 3 and the arcuate plates 81.

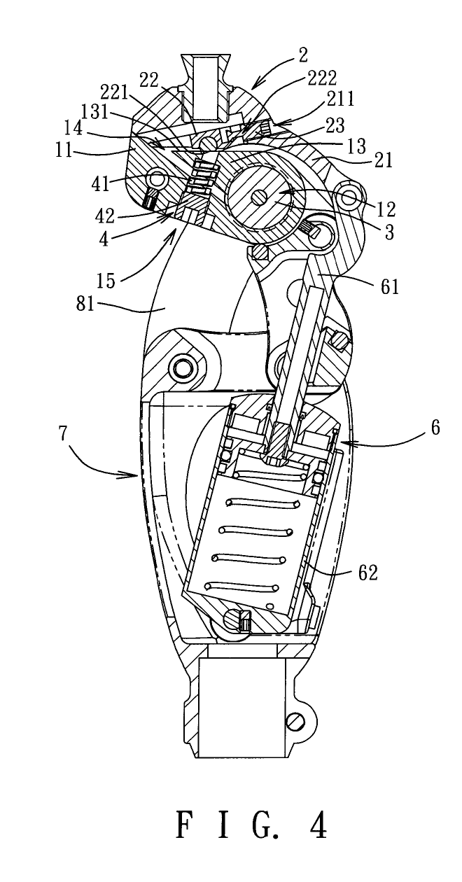

Referring to FIGS. 4 and 5, when the wearer wears the knee joint of the present invention and walks on uneven ground, the natural reflexes of the wearer may act to maintain the center of gravity of the body by shifting weight onto the knee joint. At this moment, the upper connecting unit 2 may sustain more of the weight of the user, thus causing the brake block 22 to compressibly abut against the movable plate 13 and constrict the size of the interspace 14. Due to the constriction of the interspace 14, the pivot shaft 3 is tightly clamped between the fixed seat 11 and the movable plate 13 so as to restrict rotation thereof relative to the knee member 1, thereby preventing the knee joint from rotating. Therefore, when the wearer encounters an unexpected situation that may induce a loss of balance, the knee joint of the present invention mitigates the risk of falling caused by excessive rotation of the knee joint. Additionally, the threaded relationship between the threaded portion 31 of the pivot shaft 3 and the threaded hole 12 of the knee member 1 in the present invention is able to increase friction therebetween, thereby allowing the knee member 1 to effectively restrict the rotation of the pivot shaft 3.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. A knee joint for connection with a prosthetic upper leg and a prosthetic lower leg, said knee joint comprising:
a knee member including a fixed seat, a movable plate connected integrally to said fixed seat, a threaded hole formed between said fixed seat and said movable plate, and an interspace formed between said fixed seat and said movable plate and communicating with said threaded hole, said movable plate being pressable to move toward said fixed seat and to thereby reduce a size of said interspace;
an upper connecting unit that is disposed on top of said knee member for connection with the prosthetic upper leg and that abuts against said movable plate; and
a pivot shaft that has a threaded portion inserted threadedly into said threaded hole such that said pivot shaft is rotatable relative to said knee member;
wherein, when said upper connection unit is pressed against said movable plate, said interspace is constricted so that said pivot shaft is clamped tightly between said fixed seat and said movable plate, thereby restricting a relative rotation between said knee member and said pivot shaft, said movable plate being restorable when being unpressed.

2. The knee joint as claimed in claim 1, further comprising a restoring member including a restoring spring disposed in said fixed seat to abut against said movable plate, thereby restoring said movable plate.

3. The knee joint as claimed in claim 2, wherein said knee member further includes a receiving aperture to receive said restoring member, said restoring member further including a cap secured to said receiving aperture, said restoring spring having two ends respectively abutting against said cap and said movable plate.

4. The knee joint as claimed in claim 1, wherein said upper connecting unit includes a connector for connecting to the prosthetic upper leg and a brake block disposed between said connector and said movable plate and having an abutment surface, said movable plate having a contact surface in contact with said abutment surface, wherein the size of said interspace is adjusted by displacing said brake block to move said movable plate toward or away from said fixed seat.

5. The knee joint as claimed in claim 4, wherein said upper connecting unit further includes a brake regulator extending through said connector and connecting said brake block to move said brake block and to thereby adjust said interspace.

6. The knee joint as claimed in claim 5, wherein said brake regulator is a regulating screw, said connector has a threaded slot receiving said regulating screw, said brake block further having a fixing hole receiving securely one end of said regulating screw, said brake regulator being movable within said threaded slot for adjusting movement of said brake block.
